(19) United States
(12) Patent Application Publication
(10) Pub. No.: US 2015/0216935 A1
(43) Pub. Date: Aug. 6, 2015

(54) Autologous Cell-Based Therapy for Treating Obesity

(71) Applicant: Brown University, Providence, RI (US)

(72) Inventors: Edith Mathiowitz, Brookline, MA (US); Sasha Bakhru, Providence, RI (US)

(73) Assignee: Brown University, Providence, RI (US)

(21) Appl. No.: 14/420,600

(22) PCT Filed: Aug. 8, 2013
(86) PCT No.: PCT/US2013/054094

§ 371 Date: Feb. 9, 2015

Related U.S. Application Data
(60) Provisional application No. 61/681,511, filed on Aug. 9, 2012.

(51) Int. Cl. A61K 38/18; A61K 9/70; C12N 5/077; A61K 35/35; C12N 5/0775; A61K 47/34; A61K 38/28

(52) U.S. Cl. CPC A61K 38/1858; A61K 38/18; A61K 38/1841; A61K 47/34; A61K 9/70; A61K 38/28; A61K 35/35; C12N 5/0667; C12N 5/0653; C12N 2501/155

(57) ABSTRACT

Compositions and methods for producing autologous brown adipose cells in vitro or in vivo are provided. In particular, a drug delivery device is described that recruits adipose stem cells (ASCs) to a site in the body of a subject. These ASCs may then be isolated and induced to differentiate into autologous brown adipose cells. Alternatively, the drug delivery device may also include differentiation factors that induce differentiation of the recruited ASCs into brown adipose cells in vivo. The brown adipose cells produced by these methods may be used therapeutically to treat conditions, such as obesity and diabetes.

### FIELD OF THE INVENTION

The invention is generally related to the field of autologous cells for treating diabetes, more particularly to nanoparticles or microparticles for isolating adipose stem cells.

### BACKGROUND OF THE INVENTION

Obesity is a common metabolic disorder associated with dyslipidemia, hypertension, insulin resistance, type-2 diabetes (T2DM), cardiovascular disease and increased mortality (Kelly T, et al., Int J Obes, 32:1431-37 (2008). By contributing to the burden of these chronic diseases and disabilities, obesity is connected with serious social and psychological dimensions affecting virtually all ages and socioeconomic groups. Over the last 20 years, the worldwide prevalence of obesity has increased to epidemic proportions both in the industrial world and worldwide (Kelly 2008; Mendez, M. A., et al., Am J Clin Nutr, 81:714-21 (2005); Ng, S. W., et al., Obes Rev, 12(1):1-13 (2011); Ogden, C. L., et al., JAMA, 303(3):242-9 (2010); Popkin, B. M., et al. Obesity (Silver Spring, Md.), 14:1846-1853 (2006)), and the epidemic of obesity has been accompanied by a worldwide epidemic of type 2 diabetes (commonly referred to as “T2DM”). Despite the proliferation of lifestyle modification-based strategies, the World Health Organization (WHO) predicts that the prevalence of T2DM will nearly double from 171 million in the year 2000 to 366 million in the year 2030. Perhaps even more striking, the WHO estimates that there are nearly 1 billion overweight adults worldwide with at least 300 million of them clinically obese.

Due to the severity of the consequences of obesity, liposuction has become the second most common elective plastic surgery procedure in the United States with more than 330 thousand procedures performed annually. Despite surgical intervention, it is estimated that approximately 50% of liposuction patients regain the weight within two years. Given the impact on health, quality of life, and life-span, there is an unmet clinical need for a long-lasting intervention to combat obesity and diabetes.

Obesity results from an imbalance between energy intake and energy expenditure. Both genetic and environmental factors, e.g., sedentary lifestyle and excess caloric intake, contribute to its development (Walley, A. J., et al. Nat Rev Genet, 2009. 10(19506576):431-442; Welsh, G. I., et al. Proteomics, 2004. 4:1042-1051). Increased energy (food) intake and/or decreased energy expenditure (sedentary lifestyle) results in a positive energy balance; this energy is stored in the body in the form of fat.

Two different types of fat are known to be present in the human body: 1) White adipose tissue (WAT)—the main energy store of the body and is in addition the largest endocrine organ, and 2) Brown adipose tissue (BAT)—a less abundant type specific for non-shivering thermogenesis, resulting in an increase of body heat. The developmental patterns of WAT and BAT are distinct. BAT emerges earlier than WAT during fetal development. BAT is at its greatest amount, relative to bodyweight, at birth. After birth, BAT involutes both in humans and rodents with age (Cannon, B. and J. Nedergaard. Physiol Rev, 2004. 84:277-359) and has traditionally been considered insignificant in adults. However, several reports confirmed recently that BAT exists in human adults in appreciable amounts (Cypess, A. M., et al., N Engl J Med, 2009. 360:1509-1517; Nedergaard, J., et al. Am J Physiol Endocrinol Metab, 2007. 293:444-452; Saito, M., et al., Diabetes, 2009. 58:1526-1531; van Marken Lichtenbelt, W. D., et al., N Engl J Med, 2009. 360:1500-1508; Virtanen, K. A., et al., N Engl J Med, 2009. 360:1518-1525). Recent research has indicated an important role for BAT in adult humans in the control of body temperature and adiposity (Cypess, A. M., et al., N Engl J Med, 2009. 360:1509-1517; Saito, M., et al., Diabetes, 2009. 58:1526-1531). These findings reject the notion that BAT is absent in adult humans; however, the variation between individuals is considerable (Nedergaard, J., et al. Am J Physiol Endocrinol Metab, 2007. 293:444-452). Brown adipocytes have also been observed in adults in classical white fat depots (Diehl, A. M. and J. B. Hoek. J Bioenerg Biomembr, 1999. 31:493-506). It has been suggested that white adipocytes can be transformed via genetic modifications into brown adipocytes by the peroxisome proliferation activation receptors (PPARs) and their co-factors (Tiraby, C. and D. Langin. Trends Endocrinol Metab, 2003. 14:439-441; Tiraby, C., et al. J Biol Chem, 2003. 278:33370-33376).

Enhancing brown adipose content in the body should support an increase in thermogenic energy expenditure. In particular, it has been demonstrated that the amount of BAT in human adults is inversely correlated with BMI. Furthermore, it is estimated that as little as 50 grams of BAT could account for 20% of daily energy expenditure (Rothwell, N. J. and M. J. Stock. Nature, 1979. 281(551265):31-35). So, even a small amount of BAT can yield significant increases in energy consumption. The potential to introduce even a small amount of BAT in adult humans, via autologous cellular transplantation, would provide a new approach to the treatment and/or prevention of obesity and its metabolic complications.

There is an urgent need for an abundant source of brown adipose cells for development of a cell-based therapy. The stromal compartment of mesenchymal tissues contains adipose stem cells (“ASCs”) able to both self-renew and differentiate to yield mature cells of multiple lineages, including adipose cells. ASCs may be isolated from a lipoaspirate, but less invasive methods are needed.

However, currently there is no available therapy for increasing the amount of BAT in humans.

It is therefore an object of the invention to provide methods for increasing the amount of BAT in humans. It is a further object of the inventions to provide compositions and methods for treating or preventing obesity and/or diabetes.

It is a further objection of the invention to provide methods and compositions for producing autologous brown adipose cells in effective amounts to treat or prevent conditions, such as obesity and/or diabetes.

### SUMMARY OF THE INVENTION

A drug delivery system for recruiting ASCs to a site in the body of a subject is provided. In some embodiments, the drug delivery device is used to isolate ASCs from a subject, which can be induced to differentiate into brown adipose cells ex vivo for transplantation. In other embodiments, the drug delivery device also contains differentiation factors that induce the ASCs to differentiation into brown adipose cells in vivo.

The ASC recruitment factors are releasably incorporated into the drug delivery system. In some embodiments, the drug delivery system contains or is formed from thin films, fibers and/or a plurality of particles, with one or more soluble ASC recruitment factors releasably incorporated therein. In one embodiment, the drug delivery system preferably contains a plurality of particles with one or more soluble ASC recruitment factors releasably incorporated therein. Alternatively, or in addition, the ASC recruitment factors may be releasably incorporated within a polymeric scaffold, mesh, fibers, or other structures suitable for controlled release of the ASC recruitment factors.

The one or more ASC recruitment factors are preferably released from the drug delivery system when implanted in a subject in an effective amount to recruit ASC's.

In some embodiments, the drug delivery system contains an external porous housing to facilitate removal of the ASCs. The external porous housing preferably has pores of a size sufficient to allow movement of ASCs into the system. For example, the external porous housing may be composed of a biocompatible, polymeric mesh. The external porous housing is preferably composed of a hydrophobic and non-erodable polymer. Suitable polymers for forming the external porous housing are known in the art and include polyamides, polyethylene, polypropylene, polystyrene, polyvinyl chloride, polycarbonates, poly(amino acids), polyesteramides, poly(dioxanones), poly(alkylene alkylates), polyethers, polyurethanes, polyetheresters, polyacetals, polycyanoacrylates, polysiloxanes, poly(phosphazenes), polyphosphates, polyalkylene oxalates, polyacrylonitriles, polyalkylene succinates, poly(maleic acids), polysaccharides; poly(acrylic acids), poly(methacrylic acids), and derivatives, copolymers, and blends thereof. In preferred embodiments, the polymeric mesh is composed of a biocompatible nylon.

In alternative embodiments, the drug delivery system also contains one or more brown adipogenic differentiation-inducing factors releasably incorporated therein in an effective amount for inducing differentiation of the ASCs into brown adipose cells in vivo. Examples of suitable brown adipogenic differentiation-inducing factors include bone morphogenetic protein 7 (BMP7), cyclic AMP (cAMP), retinoic acid (RA), triiodothyronine (T3), dexamethasone (Dex), growth hormone (GH), insulin, insulin-like growth factor 1 (IGF-I), or combinations thereof.

Kits are also disclosed that contain the drug delivery system and one or more brown adipogenic differentiation-inducing factors

The adipogenic differentiation-inducing factors may be releasably incorporated into the drug delivery system. In some embodiments, the drug delivery system contains or is formed from thin films, fibers and/or a plurality of particles, with one or more adipogenic differentiation-inducing factors releasably incorporated therein. In one embodiment, the drug delivery system preferably contains a plurality of particles with one or more adipogenic differentiation-inducing factors releasably incorporated therein, optionally in combination with the same or different particles that contain the ASC recruitment factors. In place of particles, the adipogenic differentiation-inducing factors and/or ASC recruitment factors may be incorporated in other drug delivery systems, such as thin films and/or fibers. In these embodiments, the brown adipogenic differentiation-inducing factors are preferably released at a delayed or slower rate than the ASC recruitment factors. For example, the particles may be biphasic or multiphasic. Alternatively, or in addition to, the adipogenic differentiation-inducing factors may be releasably incorporated within a polymeric scaffold, mesh, fibers, or other structures suitable for controlled release of the ASC recruitment factors.

The method for isolating ASCs involves introducing into the subject a drug delivery system containing an effective amount of one or more soluble ASC recruitment factors, removing the drug delivery system from the subject after a sufficient time period for ASCs to migrate into the drug delivery system, and isolating the ASCs. The method may further involve culturing the ASCs in the presence of an effective amount of one or more brown adipogenic differentiation-inducing factors to induce differentiation of the ASCs into brown adipocytes.

In one embodiment, a method for inducing brown adipose differentiation in vivo involves introducing into the subject a drug delivery system containing both an effective amount of one or more soluble ASC recruitment factors and brown adipogenic differentiation-inducing factors that are released, preferably at different times, from the drug delivery system following implantation in a subject.

In another embodiment, the method for inducing brown adipose differentiation in vivo involves administering a first drug delivery system containing an effective amount of one or more soluble ASC recruitment factors, and after a sufficient time period to recruit a sufficient amount of ASC's administering a second drug delivery system containing an effective amount of one or more brown adipogenic differentiation-inducing factors to induce differentiation of the ASCs into brown adipocytes.

The brown adipose cells produced by these methods may be used therapeutically to treat conditions, such as obesity and diabetes. In one embodiment, a method for treating obesity or diabetes in a subject involves administering to the subject an effective amount of autologous ASC-derived brown adipocytes. An alternative method involves administering to the subject an effective amount of a drug delivery system containing soluble ASC recruitment factors and brown adipogenic differentiation-inducing factors.

### DETAILED DESCRIPTION OF THE INVENTION

### I. Definitions

The term “cell” refers to isolated cells, cells from a primary culture, or cell lines unless specifically indicated.

The term “mesenchymal stem cell” or “MSC” refers to a multipotent cell found within stromal tissues (e.g., solated from placenta, adipose tissue, lung, bone marrow and blood) of an adult mammal that can differentiate into a variety of cell types, including osteoblasts (bone cells), chondrocytes (cartilage cells), and adipocytes (fat cells). Adipose tissue is one of the richest sources of MSCs. When compared to bone marrow, there are more than 500 times more stem cells in 1 gram of fat when compared to 1 gram of aspirated bone marrow.

The terms “adipose stem cell,” “adipose-derived stem cell,” and “ASC” are used interchangeably herein and refer to a MSC present within adipose tissue of an adult mammal ASCs express at least the mesenchymal stem cell markers CD34 and CD105, but may also express the mesenchymal stem cell markers CD10, CD13, CD29, CD44, CD54, CD71, CD90, CD106, CD 117, and STRO-1. ASCs are at least negative for the hematopoietic lineage marker CD36 and CD45, but are also preferably negative for the hematopoietic lineage markers CD14, CD16, CD56, CD61, CD62E, CD104, and CD 106 and for the endothelial cell (EC) markers CD31, CD 144, and von Willebrand factor. Morphologically, they are fibroblast-like and preserve their shape after expansion in vitro.

The term “brown adipose tissue” or “BAT” refers to fat in a mammal containing brown adipocytes.

The term “brown adipocyte” refers to a fat cell in a mammal containing a plurality of small lipid droplets. Brown adipocytes contain a higher number of mitochondria than white adipocytes, which contain only a single lipid droplet.

The term “brown adipogenic differentiation-inducing factor” or simply “differentiation factor” refers to an agent (e.g., protein) that directly or indirectly promotes or facilitates the differentiation of ASCs into mature brown adipocytes. The factor may be one of a combination of factors necessary to promote differentiation.

The term “controlled release” and “modified release”, are used interchangeably herein and refer to a release profile in which the active agent release characteristics of time course and/or location are chosen to accomplish therapeutic or convenience objectives not offered by conventional dosage forms such as solutions, suspensions, or promptly dissolving dosage forms. Delayed release, extended release, and pulsatile release and their combinations are examples of modified release.

The term “mean particle size” generally refers to the statistical mean particle size (diameter) of the particles in the composition. Two populations can be said to have a “substantially equivalent mean particle size” when the statistical mean particle size of the first population of nanoparticles is within 20% of the statistical mean particle size of the second population of nanoparticles; more preferably within 15%, most preferably within 10%.

The term “biocompatible” refers to a material and any metabolites or degradation products thereof that are generally non-toxic to the recipient and do not cause any significant adverse effects to the subject.

The term “biodegradable” refers to a material that will degrade or erode under physiologic conditions to smaller units or chemical species that are capable of being metabolized, eliminated, or excreted by the subject. The degradation time is a function of polymer composition and morphology. Suitable degradation times are from days to months.

The term “non-erodible” refers to a material that maintains structural integrity under physiologic conditions for at least two months.

The term “individual,” “host,” “subject,” and “patient” are used interchangeably to refer to any individual who is the target of administration or treatment. As generally used herein, the subject is a mammal, unless otherwise specified. Thus, the subject can be a human or veterinary patient.

The term “therapeutically effective amount” refers to the amount of the composition used is of sufficient quantity to ameliorate one or more causes or symptoms of a disease or disorder. Such amelioration only requires a reduction or alteration, not necessarily elimination.

The term “treatment” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder.

The terms “promote,” “promotion,” and “promoting” as used herein refer to an increase in an activity, response, condition, disease, or other biological parameter. This can include but is not limited to the initiation of the activity, response, condition, or disease. This may also include, for example, a 10% increase in the activity, response, condition, or disease as compared to the native or control level. Thus, the increase can be a 10, 20, 30, 40, 50, 60, 70, 80, 90, 100%, or any amount of increase in between as compared to native or control levels.

### II. Compositions

A. Drug Delivery System for Harvesting Adipocyte Stem Cells

The drug delivery system can have any suitable size and shape. For example, the ASC recruitment factors and/or adipogenic differentiation-inducing factors can be incorporated into and released in a controlled manner from micro- or nano-fibers, films, and/or particles. The particles can have any shape, including spherical and non-spherical shapes.

1. Materials

The particles, fibers, films, or any appropriate delivery systems are formed of any material suitable for controlled release of effective amounts and duration of these factors in physiological conditions. The particles, fibers, or films are preferably biodegradable, and preferably contain one or more biodegradable polymers, copolymers or blends thereof. Suitable biodegradable polymers include, but are not limited to, polyhydroxyacids, polyhydroxyalkanoates, poly(caprolactones), poly(orthoesters), poly(phosphazenes), polyesteramides, polyanhydrides, poly(dioxanones), poly(alkylene alkylates), poly(hydroxyacid)/poly(alkylene oxide) copolymers, poly(caprolactone)/poly(alkylene oxide) copolymers, biodegradable polyurethanes, poly(amino acids), polyetheresters, polyacetals, polycyanoacrylates, poly(oxyethylene)/poly(oxypropylene) copolymers, and derivatives, copolymers, and blends thereof. In preferred embodiments, the polyhydroxyacid is of poly(lactic acid), poly(glycolic acid), or poly(lactic acid-co-glycolic acid). Where particles, fibers, or films have a mean diameter (or other dimension) that is smaller than the mesh pore size in the polymeric mesh, the particles, fibers, or films are preferably electrostatic to prevent them from diffusing out of the mesh.

2. Methods of Manufacture

Particles useful in the drug delivery systems described herein can be prepared using any suitable method known in the art and are described in more detail below in Section 3. For fiber formation, any kind of solvent extrusion, wet spinning, melt extrusion, or dry spinning method can be used to form fibers having suitable dimensions and properties. For film formation, any kind of film casting method can be used to form films having suitable dimensions and properties.

The larger delivery system could be later cut to the desired size so that the film or fiber could fit inside the mesh and release the drug in suitable manner.

Alternatively, the films may be cut to the desired size and implanted directly in the region of interest (e.g., fat tissue).

3. Particles

The drug delivery system preferably contains a plurality of particles, which provide controlled release of ASC recruitment factors and/or adipogenic differentiation-inducing factors.

a. Sizes

The particles may be of any size and material suitable for release of an effective amount and duration of the disclosed factors. For example, the particles may have average particle size of from 1 nm to 1 mm, preferably from 1 nm to 100 μm, more preferably from 10 nm to 10 μm. In preferred embodiments, the particles are nanoparticles, having a size range from about 10 nm to 1 micron, preferably from about 10 nm to about 0.1 microns. In particularly preferred embodiments, the particles have a size range from about 500 to about 600 nm. The particles can have any shape but are generally spherical in shape.

b. Methods of Manufacture

Common microencapsulation techniques include, but are not limited to, spray drying, interfacial polymerization, hot melt encapsulation, phase separation encapsulation (spontaneous emulsion microencapsulation, solvent evaporation microencapsulation, and solvent removal microencapsulation), coacervation, low temperature microsphere formation, and phase inversion nanoencapsulation (PIN). A brief summary of these methods is presented below.

In certain embodiments, the nanoparticles incorporated in the compositions discussed herein are multi-walled nanoparticles. Multi-walled nanoparticles useful in the compositions disclosed herein can be prepared, for example, using “sequential phase inversion nanoencapsulation” (sPIN).

1. Spray Drying

Methods for forming microspheres/nanospheres using spray drying techniques are described in U.S. Pat. No. 6,620,617, to Mathiowitz et al. In this method, the polymer is dissolved in an organic solvent such as methylene chloride or in water. A known amount of one or more active agents to be incorporated in the particles is suspended (in the case of an insoluble active agent) or co-dissolved (in the case of a soluble active agent) in the polymer solution. The solution or dispersion is pumped through a micronizing nozzle driven by a flow of compressed gas, and the resulting aerosol is suspended in a heated cyclone of air, allowing the solvent to evaporate from the microdroplets, forming particles. Microspheres/nanospheres ranging between 0.1-10 microns can be obtained using this method.

2. Interfacial Polymerization

Interfacial polymerization can also be used to encapsulate one or more active agents. Using this method, a monomer and the active agent(s) are dissolved in a solvent. A second monomer is dissolved in a second solvent (typically aqueous) which is immiscible with the first. An emulsion is formed by suspending the first solution through stirring in the second solution. Once the emulsion is stabilized, an initiator is added to the aqueous phase causing interfacial polymerization at the interface of each droplet of emulsion.

3. Hot Melt Micro En Capsulation

Microspheres can be formed from polymers such as polyesters and polyanhydrides using hot melt microencapsulation methods as described in Mathiowitz et al., Reactive Polymers, 6:275 (1987). In this method, the use of polymers with molecular weights between 3-75,000 daltons is preferred. In this method, the polymer first is melted and then mixed with the solid particles of one or more active agents to be incorporated that have been sieved to less than 50 microns. The mixture is suspended in a non-miscible solvent (like silicon oil), and, with continuous stirring, heated to 5° C. above the melting point of the polymer. Once the emulsion is stabilized, it is cooled until the polymer particles solidify. The resulting microspheres are washed by decanting with petroleum ether to give a free-flowing powder.

4. Phase Separation Microencapsulation

In phase separation microencapsulation techniques, a polymer solution is stirred, optionally in the presence of one or more active agents to be encapsulated. While continuing to uniformly suspend the material through stirring, a nonsolvent for the polymer is slowly added to the solution to decrease the polymer's solubility. Depending on the solubility of the polymer in the solvent and nonsolvent, the polymer either precipitates or phase separates into a polymer rich and a polymer poor phase. Under proper conditions, the polymer in the polymer rich phase will migrate to the interface with the continuous phase, encapsulating the active agent(s) in a droplet with an outer polymer shell.

i. Spontaneous Emulsion Microencapsulation

Spontaneous emulsification involves solidifying emulsified liquid polymer droplets formed above by changing temperature, evaporating solvent, or adding chemical cross-linking agents. The physical and chemical properties of the encapsulant, as well as the properties of the one or more active agents optionally incorporated into the nascent particles, dictates suitable methods of encapsulation. Factors such as hydrophobicity, molecular weight, chemical stability, and thermal stability affect encapsulation.

ii. Solvent Evaporation Microencapsulation

Methods for forming microspheres using solvent evaporation techniques are described in E. Mathiowitz et al., J. Scanning Microscopy, 4:329 (1990); L. R. Beck et al., Fertil. Steril., 31:545 (1979); L. R. Beck et al Am J Obstet Gynecol 135(3) (1979); S. Benita et al., J. Pharm. Sci., 73:1721 (1984); and U.S. Pat. No. 3,960,757 to Morishita et al. The polymer is dissolved in a volatile organic solvent, such as methylene chloride. One or more active agents to be incorporated are optionally added to the solution, and the mixture is suspended in an aqueous solution that contains a surface active agent such as poly(vinyl alcohol). The resulting emulsion is stirred until most of the organic solvent evaporated, leaving solid microspheres/nanospheres. This method is useful for relatively stable polymers, such as polyesters and polystyrene. However, labile polymers, such as polyanhydrides, may degrade during the fabrication process due to the presence of water. For these polymers, some of the following methods performed in completely anhydrous organic solvents are more useful.

iii. Solvent Removal Microencapsulation

The solvent removal microencapsulation technique is primarily designed for polyanhydrides and is described, for example, in WO 93/21906 to Brown University Research Foundation. In this method, the substance to be incorporated is dispersed or dissolved in a solution of the selected polymer in a volatile organic solvent, such as methylene chloride. This mixture is suspended by stirring in an organic oil, such as silicon oil, to form an emulsion. Microspheres that range between 1-300 microns can be obtained by this procedure. Substances which can be incorporated in the microspheres include pharmaceuticals, pesticides, nutrients, imaging agents, and metal compounds.

5. Coacervation

Encapsulation procedures for various substances using coacervation techniques are known in the art, for example, in GB-B-929 406; GB-B-929 40 1; and U.S. Pat. Nos. 3,266,987, 4,794,000, and 4,460,563. Coacervation involves the separation of a macromolecular solution into two immiscible liquid phases. One phase is a dense coacervate phase, which contains a high concentration of the polymer encapsulant (and optionally one or more active agents), while the second phase contains a low concentration of the polymer. Within the dense coacervate phase, the polymer encapsulant forms nanoscale or microscale droplets. Coacervation may be induced by a temperature change, addition of a non-solvent or addition of a micro-salt (simple coacervation), or by the addition of another polymer thereby forming an interpolymer complex (complex coacervation).

6. Low Temperature Casting of Microspheres

Methods for very low temperature casting of controlled release microspheres are described in U.S. Pat. No. 5,019,400 to Gombotz et al. In this method, a polymer is dissolved in a solvent optionally with one or more dissolved or dispersed active agents. The mixture is then atomized into a vessel containing a liquid non-solvent at a temperature below the freezing point of the polymer-substance solution which freezes the polymer droplets. As the droplets and non-solvent for the polymer are warmed, the solvent in the droplets thaws and is extracted into the non-solvent, resulting in the hardening of the microspheres.

7. Phase Inversion Nanoencapsulation (PIN)

Nanoparticles can also be formed using the phase inversion nanoencapsulation (PIN) method, wherein a polymer is dissolved in a “good” solvent, fine particles of a substance to be incorporated, such as a drug, are mixed or dissolved in the polymer solution, and the mixture is poured into a strong non-solvent for the polymer, to spontaneously produce, under favorable conditions, polymeric microspheres, wherein the polymer is either coated with the particles or the particles are dispersed in the polymer. See, e.g., U.S. Pat. No. 6,143,211 to Mathiowitz, et al. The method can be used to produce monodisperse populations of nanoparticles and microparticles in a wide range of sizes, including, for example, about 100 nanometers to about 10 microns.

Advantageously, an emulsion need not be formed prior to precipitation. The process can be used to form microspheres from thermoplastic polymers.

8. Sequential Phase Inversion Nanoencapsulation (sPIN)

Multi-walled nanoparticles can also be formed by a process referred to herein as “sequential phase inversion nanoencapsulation” (sPIN). sPIN is particularly suited for forming monodisperse populations of nanoparticles, avoiding the need for an additional separations step to achieve a monodisperse population of nanoparticles.

In sPIN, a core polymer is dissolved in a first solvent. The active agent is dissolved or dispersed in a core polymer solvent. The core polymer, core polymer solvent, and agent to be encapsulated form a mixture having a continuous phase, in which the core polymer solvent is the continuous phase. The shell polymer is dissolved in a shell polymer solvent, which is a non-solvent for the core polymer. The solutions of the core polymer and shell polymer are mixed together. The resulting decreases the solubility of the core polymer at its cloud point due to the presence of the shell polymer solvent results in the preferential phase separation of the core polymer and, optionally, encapsulation of the agent. When a non-solvent for the core polymer and the shell polymer is added to this unstable mixture, the shell polymer engulfs the core polymer as phase inversion is completed to form a double-walled nanoparticle.

sPIN provides a one-step procedure for the preparation of multi-walled particles, such as double-walled nanoparticles, which is nearly instantaneous, and does not require emulsification of the solvent. Methods for forming multi-walled particles are disclosed in U.S. Publication No. 2012-0009267 to Cho, et al. The disclosure of which is incorporated herein by reference.

The number of walls is dependent on identifying suitable polymer-solvent pairs. For example, to form a triple-walled nanoparticle, a core polymer is dissolved in a core polymer solvent to form a core polymer solution, where the core polymer solvent is a solvent for the core polymer, a second polymer and the shell polymer. The second polymer is dissolved in a polymer solvent to form a second polymer solution, where the second polymer solvent is a solvent for the second polymer but is not a solvent for the core polymer. The shell polymer is dissolved in a shell polymer solvent to form a shell polymer solution, where the shell polymer solvent is a solvent for the shell polymer, but is not a solvent for the core polymer or the second polymer.

The core polymer solution is added to the second polymer solution, optionally in the presence of an agent to be encapsulated. The resulting decrease in the solubility of the core polymer due to the presence of the second polymer solvent results in the preferential phase separation of the core polymer and, if desired, encapsulation of the agent. Then the shell polymer solution is added to this mixture. The resulting decrease in the solubility of the second polymer due to the presence of the shell polymer solvent results in the preferential phase separation of the second polymer which encapsulates the core polymer. Finally, a non-solvent for the core polymer, second polymer, and shell polymer can be added to this mixture. The resulting decrease in the solubility of the shell polymer due to the presence of the non-solvent results in the preferential phase separation of the shell polymer thereby forming triple-walled nanoparticles.

An alternative method for forming multi-walled nanoparticles having three or more layers involves adding the non-solvent after the second polymer solution is mixed with the core polymer solution. In this embodiment, the core polymer solution, second polymer solution and shell solution are formed as described above. Then the core polymer solution and second polymer solution are mixed. Next the non-solvent is added, thereby forming double-walled nanoparticles in the solvent-non-solvent mixture. Finally, the third polymer solution is added to this mixture, to form triple-walled nanoparticles.

The above-described method can be further modified by selecting appropriate solvents for the polymers and a non-solvent for all of the polymers, as described above with respect to double- and triple-walled nanoparticles, to include additional walls in the multi-walled nanoparticles.

In one embodiment, the multi-walled nanoparticles can be formed in the absence of a non-solvent, and/or where the second polymer solvent is the same as the core polymer solvent. For example, precipitation of the core polymer can be controlled by change in temperature of the operating conditions. Alternatively precipitation of one of the polymers can be controlled by the addition of one or more excipients that act as precipitating agents for the core polymer, second polymer, and/or shell polymer. The precipitating agent depends on the polymers and solvents used. Exemplary agents include salts.

4. Mesh

In some embodiments, the drug delivery system also contains an external porous housing to facilitate removal of the ASCs. The external porous housing preferably has pores of a size sufficient to allow movement of ASCs into the system. Exemplary pore sizes include at least 3 microns, at least 5 microns, optionally ranging from about 3 to 5 microns, at least 10 microns, at least 20 microns, at least 30 microns, at least 40 microns, and at least 50 microns. The upper limit of the pore sizes typically ranges from 100 to 999 microns, in some embodiments the upper limit is about 100 microns, about 200 microns, about 300 microns, about 400 microns, about 500 microns, about 600 microns, about 700 microns, about 800 microns, about 900 microns, or less than about 1000 microns. Preferably the size of the pores range from about 10 microns to about 500 microns. The pores may be of regular or irregular shape. The pores may be generally circular, although the shape of the pores is not so limited since it is possible for most cells to deform their shape into order to move into the implant.

The external porous housing may be composed of a polymeric mesh. The polymeric mesh preferably is formed from one or more hydrophobic and non-erodable polymer(s). Suitable polymers for forming the external porous housing are known in the art and include polyamides, polyethylene, polypropylene, polystyrene, polyvinyl chloride, polycarbonates, poly(amino acids), polyesteramides, poly(dioxanones), poly(alkylene alkylates), polyethers, polyurethanes, polyetheresters, polyacetals, polycyanoacrylates, polysiloxanes, poly(phosphazenes), polyphosphates, polyalkylene oxalates, polyacrylonitriles, polyalkylene succinates, poly(maleic acids), polysaccharides; poly(acrylic acids), poly(methacrylic acids), and derivatives, copolymers, and blends thereof. In preferred embodiments, the polymeric mesh is composed of a nylon.

B. ASC Recruitment Factors

In order to participate in repair and regeneration, ASCs have to be mobilized and then migrate to the target sites and integrate with the local tissues. The mechanisms for ASCs to migrate to injured tissues include chemoattractants, paracrine factors, membrane receptors, and intracellular signaling molecules. Extracellular matrix and biophysical factors play important role in guiding migration of ASCs.

In some embodiments, one or more suitable ASC recruitment factors are incorporated into and administered via the drug delivery systems described herein. In some embodiments, the ASC recruitment factors are soluble. Preferably the ASC recruitment factor is SDF-1, a PDGF (e.g., PDGF-BB), a TGFβ, or a combination thereof. The ASC recruitment factors are released from the drug delivery system for at least 7 days, preferably at least 14 days, more preferably at least 21 days following implantation in a subject.

1. SDF-1

Stromal-derived factor 1 (SDF-1) is small cytokine belonging to the chemokine family that is involved in MSC migration. SDF-1 is officially designated Chemokine (C-X-C motif) ligand 12 (CXCL12). SDF-1 is produced in two forms, SDF-1α/CXCL12a and SDF-1β/CXCL12b, by alternate splicing of the same gene.

SDF-1 was first identified as a lymphocyte and monocyte specific chemo-attractant under both normal and inflammatory conditions. Subsequently it has been demonstrated that MSCs express CXCR4, the receptor for SDF-1, and therefore SDF-1/CXCR4 axis has been implicated in the migration of MSC in a series of studies. Those studies suggest that SDF-1/CXCR4 axis was required for migration of human bone marrow MSCs and cord blood MSCs. CXCR4 antagonist AMD3100 significantly inhibited chemotaxis of MSCs toward SDF-1. Rat bone marrow MSCs were shown to migrate towards SDF-1 gradient in a dose-dependent manner. In a rat model, SDF-1-CXCR4 was shown to mediate homing of transplanted MSCs to injured sites in the brain.

SDF-1 induction stimulates a number of protective anti-inflammatory pathways, causes the down regulation of pro-inflammatory mediators and can prevent cell death. Furthermore, SDF-1 recruits stem cells to the site of tissue damage, which promotes tissue preservation and blood vessel development.

In preferred embodiments, the SDF-1 is recombinant human SDF-1α, SDF-1β, or a conservative variant thereof. Recombinant SDF-1 proteins are commercially available from, for example, PROSPEC (East Brunswick, N.J.) and R&D SYSTEMS (Minneapolis, Minn.).

2. PDGF

Several growth factors, such as platelet-derived growth factor (PDGF), and their receptors may be involved in MSC migration. MSCs express receptors for those growth factors at a moderate to high level, including platelet-derived growth factor receptor (PDGF-R), insulin-like growth factor 1 receptor (IGF 1-R), epidermal growth factor receptor (EGF-R) and Ang-1 receptor. There are five different isoforms of PDGF that activate cellular response through two different receptors. Known ligands include A (PDGFA), B (PDGFB), C (PDGFC), and D (PDGFD), and an AB heterodimer. PDGF signaling network involves two receptors, PDGFRα and PDGFRβ. All PDGFs function as secreted, disulphide-linked homodimers, but only PDGFA and B can form functional heterodimers.

The different ligand isoforms have variable affinities for the receptor isoforms, and the receptor isoforms may variably form hetero- or homo-dimers. This leads to specificity of downstream signaling. PDGF-BB is the highest-affinity ligand for the PDGFRβ.

In preferred embodiments, the PDGF is a recombinant human PDGF, such as recombinant human PDGF-BB. Recombinant PDGF proteins are commercially available from, for example, MILLIPORE (Billerica, Mass.) and R&D SYSTEMS (Minneapolis, Minn.).

3. TGFβ

Transforming growth factor-β (TGF-β) signaling pathway is involved in MSC migration. TGF-β is a secreted protein that exists in at least three isoforms called TGF-1β, TGF-1β and TGF-1β. This pathway involves phosphorylation of receptor-regulated SMADs (R-SMADs) by TbRI. SMAD2, SMAD3 and SMAD4, downstream of TbRI, are each required for TGF-β-induced MSC migration.

In preferred embodiments, the TGF-β is a recombinant human TGF-β. Recombinant TGF-β proteins are commercially available from, for example, INVITROGEN (Grand Island, N.Y.) and R&D SYSTEMS (Minneapolis, Minn.).

C. Brown Adipogenic Differentiation-Inducing Factors

Adipocytes are derived from multipotent MSCs in a process involving commitment to the adipocyte lineage to form preadipocytes followed by terminal differentiation of the committed preadipocytes into adipocytes. The process is regulated via complex interaction of external and internal clues.

Brown adipose tissue (BAT) contains a protein named uncoupling protein (UCP). UCP is organized in the inner mitochondrial membrane and functions to dissipate the H1 electrochemical potential, thereby uncoupling fuel oxidation from the phosphorylation of ADP. UCP is expressed only in brown adipocytes and is responsible for the unique thermogenic properties of this cell type. Therefore, UCP expression is a marker of brown adipogenesic differentiation.

In some embodiments, one or more suitable brown adipogenic differentiation-inducing factors are incorporated into and administered via the drug delivery systems described herein. In some embodiments, the differentiation-inducing factor is: a PPARγ activator, modulator, or inhibitor (e.g., rosiglitazone), a PPARα activator or modulator (e.g., GW9578), a PPARδ activator or modulator (e.g., GW501516 or GW0742), a dual PPARα and PPARδ activator or modulator, a pan-PPAR (α, β, γ) activator or modulator (e.g., GW4148), a PDE4 inhibitor (e.g., rolipram or IBMX), a PDE7 inhibitor (e.g., BMS 586353 or BRL 50481 or IBMX), a NRIP1 (RIP140) inhibitor, a PTEN inhibitor (e.g., potassium bisperoxo(bipyridine)oxovanadate or dipotassium bisperoxo(5-hydroxypyridine-2-carboxyl)oxovanadate), an α₁-adrenergic full or partial agonist (e.g., phenylephrine or cirazoline), an RxRα activator or modulator (e.g., LGD1069 (Targretin) or 9-cis retinoic acid), a PGC-1α activator, a PGC-1β inhibitor or activator, adiponectin or an activator of adiponectin receptor AdipoR1 and/or AdipoR2, an NOS inhibitor or activator (e.g., 2-Ethyl-2-thiopseudourea or NG-nitro-L-arginine methyl ester (L-NAME) or adenosine), a Rho kinase-ROCK inhibitor (e.g., fasudil), BDNF, a monoamine oxidase (MAO) A inhibitor and/or a MAO B inhibitor (e.g., isocarboxazid, moclobemide, selegiline), an activator of SRC, an inhibitor of EGFR (e.g., erlotinib or ZD1839-gefinitib or Argos protein), an inhibitor of FAAH (e.g., URB597), an inhibitor of MAPK 1 (e.g., PD98059) or 2 (e.g., PD98059) or 4 or 5 or 7 or 8 (e.g., PD98059), an inhibitor of CDK9 (e.g., 1,5,6,7-Tetrahydro-2-(4-pyridinyl)-4H-pyrrolo[3,2-c]pyridin-4-one hydrochloride), a TGR5 agonist (e.g., oleanolic acid), an AMPK activator (e.g., AICAR), BMP-7, an mTOR inhibitor (e.g., rapamycin), an adenylate cyclase activator (e.g., forskolin), or combinations of any of the foregoing.

In preferred embodiments, the differentiation-inducing factor is Bone morphogenetic protein 7 (BMP7), cyclic AMP (cAMP), retinoic acid (RA), Triiodothyronine (T3), glucocorticoids (dexamethasone), growth hormone, insulin, Insulin-like Growth Factor 1 (IGF-I), or any combination thereof.

1. Bone Morphogenetic Protein 7 (BMP7)

Bone morphogenetic proteins (BMPs) are members of the transforming growth factor-β superfamily and control multiple key steps of embryonic development and differentiation, including adipogenesis.

While some members of the family of bone morphogenetic proteins (BMP) support white adipocyte differentiation, BMP-7 singularly promotes differentiation of brown preadipocytes. BMP-7 triggers commitment of mesenchymal progenitor cells to a brown adipocyte lineage, and implantation of these cells into nude mice results in development of adipose tissue containing mostly brown adipocytes.

In preferred embodiments, the BMP-7 is a recombinant human BMP-7. Recombinant BMP-7 proteins are commercially available from, for example, INVITROGEN (Grand Island, N.Y.) and R&D SYSTEMS (Minneapolis, Minn.).

2. Cyclic AMP (cAMP) Agonist

Cyclic AMP (cAMP)-dependent processes are pivotal during the early stages of adipocyte differentiation. Factors that increase cellular cyclic AMP (cAMP), such as isobutylmethylxanthine (IBMX) or forskolin, strongly accelerate the initiation of the differentiation program. cAMP is synthesised from ATP by adenylyl cyclase located on the inner side of the plasma membrane. Adenylyl cyclase is activated by a range of signaling molecules through the activation of adenylyl cyclase stimulatory G (Gs)-protein-coupled receptors. Exemplary cAMP agonists include phosphodiesterase inhibitors (IBMX), dibutyryl cAMP, theophylline, prostaglandin E1, forskolin, 8-(4-chlorophenylthio)-cAMP (CPT-cAMP)

3. Retinoic Acid Receptor Agonists

Retinoic acid (RA) is a metabolite of vitamin A (retinol) that mediates the functions of vitamin A required for growth and development. All-trans-retinoic acid is a transcriptional activator of UCP1 gene expression in brown adipocytes. RA has been shown to promote differentiation of stem cells into adipocytes. Retinoic acid receptor agonists may therefore be used as a brown adipogenic differentiation-inducing factor.

Retinoic acid acts by binding to the retinoic acid receptor (RAR), which is bound to DNA as a heterodimer with the retinoid X receptor (RXR) in regions called retinoic acid response elements (RAREs). Binding of the retinoic acid ligand to RAR alters the conformation of the RAR, which affects the binding of other proteins that either induce or repress transcription of a nearby gene. Retinoic acid receptors mediate transcription of different sets of genes controlling differentiation of a variety of cell types, thus the target genes regulated depend upon the target cells. In some cells, one of the target genes is the gene for the retinoic acid receptor itself (RAR-beta in mammals), which amplifies the response.

Retinoic acid can be produced in the body by two sequential oxidation steps that convert retinol to retinaldehyde to retinoic acid. The enzymes that generate retinoic acid for control of gene expression include retinol dehydrogenases (i.e. Rdh10) that metabolize retinol to retinaldehyde, and retinaldehyde dehydrogenases (Raldh1, Raldh2, and Raldh3) that metabolize retinaldehyde to retinoic acid.

Retinoic acid receptor agonists are commercially available and include a retinoic acid or an all-trans retinoic acid.

4. Triiodothyronine (T3)

Triiodothyronine (T3) is a thyroid hormone (TH) that actively stimulates UCP in brown fat under minimal sympathetic activity. Production of T3 and its prohormone thyroxine (T4) is activated by thyroid-stimulating hormone (TSH), which is released from the pituitary gland.

In preferred embodiments, the T3 is a recombinant human T3. Recombinant T3 proteins are commercially available from, for example, AMSBIO (Lake Forest, Calif.).

5. Dexamethasone (Dex)

Dexamethasone is a potent synthetic member of the glucocorticoid class of steroid drugs. A combination of dexamethasone and insulin has been shown to promote differentiation of ASCs. Dexamethasone, and other suitable glucocorticoids, are commercially available.

6. Growth Hormone (GH)

Growth hormone (GH) is a peptide hormone that stimulates growth, cell reproduction and regeneration. GH is strictly required in the conversion of preadipocytes to adipocytes and is thought to play a role in priming the cells to become responsive to insulin and insulin-like growth factor-I (IGF-I). GH also stimulates adipogenesis, although the role of GH is not exclusive.

Commercially available recombinant human growth hormones (rHGH) included NUTROPIN (Genentech), HUMATROPE (Lilly), GENOTROPIN (Pfizer), NORDITROPIN (Novo), SAIZEN (Merck Serono), and OMNITROPE (Sandoz).

7. Insulin and Insulin-Like Growth Factor 1 (IGF-I)

Brown adipose tissue plays an important role in obesity, insulin resistance, and diabetes. The transition from brown preadipocytes to mature adipocytes is mediated in part by insulin receptor substrate (IRS)-1 and the cell cycle regulator protein necdin. Insulin/IGF-I act through IRS-1 phosphorylation to stimulate differentiation of brown preadipocytes via two complementary pathways: 1) the Ras-ERK1/2 pathway to activate CREB and 2) the phosphoinositide 3 kinase-Akt pathway to deactivate FoxO1. These two pathways combine to decrease necdin levels and permit the clonal expansion and coordinated gene expression necessary to complete brown adipocyte differentiation.

In preferred embodiments, the insulin is a recombinant human insulin. Recombinant insulin proteins are commercially available from, for example, Eli Lilly (Indianapolis, Ind.) under the brand name HUMULIN. HUMULIN is a short-acting insulin that has a relatively short duration of activity as compared with other insulins. HUMULIN N is an intermediate-acting insulin with a slower onset of action and a longer duration of activity than HUMULIN R.

In preferred embodiments, the IGF-I is a recombinant human IGF-I. Recombinant IGF-I proteins are commercially available from, for example, BD Biosciences (San Jose, Calif.) and R&D SYSTEMS (Minneapolis, Minn.).

D. Excipients

The drug delivery system typically also includes pharmaceutically acceptable excipients, such as diluents, preservatives, binders, lubricants, disintegrators, swelling agents, fillers, stabilizers, and combinations thereof.

Excipients also include all components of any coating formed around the disclosed particles, which may include plasticizers, pigments, colorants, stabilizing agents, and glidants.

E. Pharmaceutically Acceptable Carriers

The drug delivery system typically also includes a pharmaceutically acceptable carrier. For embodiments in which the drug delivery system includes a plurality of particles, fibers and/or films which provide controlled release of ASC recruitment factors and/or adipogenic differentiation-inducing factors, any pharmaceutically acceptable carrier may be used. Exemplary carriers include water for injection, sterile water, saline, buffered saline (e.g. phosphate buffered saline), and solutions or suspensions containing one or more excipients.

For embodiments in which the ASCs are administered to the patient, the carrier is typically a buffered solution (e.g. saline) or suspension such as phosphate buffered saline (PBS).

The carrier may also contain stabilizing agents, such as mall molecular weight materials that stabilize the specific proteins, such as polyols, such as glycerol, xylitol, sorbitol, inositol, and mannitol; and sugars, such as sucrose, lactose, trehalose, maltose, glucose, preferably trehalose ((α-D-glucopyranosyl(1→1)-α-D-glucopyranoside); and glycans, such as dextran.

### III. Methods

A. In Vivo Recruitment of Autologous ASCs

The stromal compartment of mesenchymal tissues contains adult stem cells, able to both self-renew and differentiate to yield mature cells of multiple lineages. These mesenchymal stem cells (MSCs) have been identified in a variety of mesodermal tissues including bone marrow (Friedenstein, A. J., et al. Exp Hematol, 1974. 2(2):83-92; Friedenstein, A. J., et al. Exp Hematol, 1976. 4(5):267-74), cardiac tissue (Beltrami, A. P., et al. Cell, 2003. 114(6):763-76), perichondrial tissue (Arai, F., et al. J Exp Med, 2002. 195(12):1549-63; Dounchis, J. S., et al. J Orthop Res, 1997. 15(6):803-7), and recently adipose tissue (Guilak, F., et al. J Cell Physiol, 2006. 206(1):229-37; Zuk, P. A., et al. Tissue Eng, 2001. 7(2):211-28). These cells share several key properties, including an ability to adhere to tissue culture plastic, forming fibroblastic-like colonies (CFU-F), extensive proliferative capacity, ability to differentiate into several mesodermal lineages including bone, muscle, cartilage and fat, and express several common cell surface antigens (Choi, Y. S., et al. J Cell Mol Med, 2010. 14(4):878-89).

In particular, mammalian adipose tissue contains a larger fraction of MSCs (a.k.a. adipose stem cells (ASCs)) than cord blood and bone marrow (Kern, S., et al. Stem Cells, 2006. 24(5):1294-301; Fraser, J. K., et al. Trends Biotechnol, 2006. 24(4):150-4). These ASCs exhibit a CD45⁻/CD31⁻/CD34⁺/CD105⁺ surface phenotype; and, freshly isolated from adipose tissue form CFU-F, proliferate and can be differentiated towards several lineages including osteogenic (Elabd, C., et al. Biochem Biophys Res Commun, 2007. 361(2):342-8; Darling, E. M., et al. J Biomech, 2008. 41(17825308):454-464; Scheideler, M., et al., BMC Genomics, 2008. 9(18637193):340-340), chrondrogenic (Darling, E. M., et al. J Biomech, 2008. 41(17825308):454-464; Erickson, G. R., et al. Biochemical & Biophysical Research Communications, 2002. 290(2):763-9), adipogenic (Darling, E. M., et al. J Biomech, 2008. 41(17825308):454-464; Scheideler, M., et al., BMC Genomics, 2008. 9(18637193):340-340; Rodriguez, A. M., et al. Biochem Biophys Res Commun, 2004. 315(2):255-63), and brown adipogenic (Elabd, C., et al. Stem Cells, 2009. 27(11):2753-60) lineages.

In one embodiment, the method for isolating ASCs from adipose tissue of a subject includes introducing into the subject the drug delivery system containing an effective amount of one or more soluble ASC recruitment factors to attract ASC's to the drug delivery system, removing the drug delivery system from the subject after a sufficient time period for ASCs to migrate into the drug delivery system, and isolating the ASCs. The method may further involve culturing the ASCs in the presence of an effective amount of one or more brown adipogenic differentiation-inducing factors to induce differentiation of the ASCs into brown adipocytes.

Alternative methods are for inducing brown adipose differentiation in vivo are also disclosed. These methods include introducing into the subject one or more drug delivery systems containing an effective amount of one or more soluble ASC recruitment factors and brown adipogenic differentiation-inducing factors that are released from the drug delivery system following administration to a subject. Preferably the drug delivery system contains both the ASC recruitment factors and brown adipogenic differentiation-inducing factors and is administered in a single administration. In this embodiment, the drug delivery system may first release the ASC recruitment factors, such as within 3 to 28 days, preferably 7 to 14 days following administration of the drug delivery system, and subsequently release the brown adipogenic differentiation factors, such as after 3 to 28 days, preferably after 7 to 14 days following administration of the drug delivery system. Optionally two or more delivery systems are administered in two or more separate administrations. In some embodiments in which the drug delivery includes a mesh, following administration of the mesh, it may be removed after one, two, three, or four weeks, or longer following administration.

B. Administration of Drug Delivery System

The disclosed drug delivery system may be administered to a subject using routine methods. In some embodiments, the plurality of particles are injected into adipose tissue of the subject, e.g., using a syringe. In other embodiments, the drug delivery device is implanted surgically in the adipose tissue. The fibers or film may be injected using suitable devices or surgically implanted, such as by small (minimally invasive) surgery. If the drug delivery device includes a mesh, it will typically be surgically implanted, such as by small (minimally invasive) surgery.

The drug delivery system is preferably administered to a site in the subject's body with high levels of ASC. Suitable sites include but are not limited to: under the skin, such as in the hypodermis; around the kidneys and in the buttocks; in the abdominal cavity, visceral fat is generally packed between the organs (e.g. stomach, liver, intestines, kidneys, etc.); around the heart; around the kidneys; and around the joints. Preferably, drug delivery systems containing one or more ASC recruitment factors are administered to a site containing white adipose tissue, such as a site containing omental fat (i.e. fatty layer of tissue located inside the belly), or subcutaneous fat.

1. Isolation and Purification of ASCs

ASCs that are recruited by the drug delivery system may be extracted from the subject using any suitable extraction method. Preferably the extraction method is minimally invasive. In some embodiments, the drug delivery system contains a plurality of particles within an external porous housing that traps ASCs recruited by recruitment factors. In these embodiments, the ASCs are removed by surgical removal of the external porous housing.

ASCs may also be removed by isolation of recruited cells at the injection/implantation site. In some embodiments, these cells are isolated by surgical resection or by aspiration.

C. Brown Adipogenic Differentiation of ASCs

1. Cell Culture

Isolated ASCs may be expanded and induced to differentiate in vitro into brown adipose cells. This method involves culturing the ASCs in a culture medium suitable for the growth, maintenance, and/or differentiation of multipotent stem cells. Once the ASCs have been expanded, the medium may then be supplemented with reagents that promote adipogenesis differentiation.

Culture media optimized for mesenchymal stem cell expansion and differentiation are commercially available. For example, STEMPRO MSC SFM (GIBCO, Grand Island, N.Y.) is a serum-free medium specially formulated for the growth and expansion of human mesenchymal stem cells. STEMPRO Adipogenesis Differentiation Kit (GIBCO, Grand Island, N.Y.) contains all reagents required for inducing MSCs to be committed to the adipogenesis pathway and generate adipocytes.

In addition, brown adipogenic differentiation-inducing factors may be added to the culture medium to facilitate/promote adipogenic differentiation. Examples of suitable brown adipogenic differentiation-inducing factors include bone morphogenetic protein 7 (BMP7), cyclic AMP (cAMP), retinoic acid (RA), triiodothyronine (T3), dexamethasone (Dex), growth hormone (GH), insulin, insulin-like growth factor 1 (IGF-I), or combinations thereof. Kits are also disclosed that contain the disclosed drug delivery system and one or more brown adipogenic differentiation-inducing factors.

2. Cell Characterization and Purification

Brown adipose cells may be characterized and purified from the cell cultures using routine methods. For example, in some embodiments, cells are selected that have a multivacuolar lipid depot and numerous typical mitochondria with dense cristae. In some embodiments, UCP gene expression may be used to identify brown adipocytes.

D. Cell Based Treatment with Brown Adipocytes

Brown adipose cells produced by the disclosed methods may be administered in a therapeutically effective amount to a subject in need thereof to treat conditions, such as obesity and diabetes. A method for treating obesity or diabetes in a subject involves administering to the subject an effective amount of autologous ASC-derived brown adipocytes. An effective amount of brown adipose cells can be determined for each patient. Typical amounts are at least 1M, more preferably greater than 10M, and optionally up to hundreds of millions brown adipose cells will be administered to the subject.

The brown adipose cells may be administered by any suitable means, including injection and implantation. In one embodiment, the brown adipose cells are implanted surgically, e.g., by laparoscopy, within a subject in need thereof using routine methods. In preferred embodiments, the cells are injected into a site in the subject.

The brown adipose cells are preferably implanted within adipose tissue of the subject. For example, the cells may be implanted within subcutaneous adipose tissue (SAT). Suitable sites include but are not limited to: under the skin, such as in the hypodermis; around the kidneys and in the buttocks; in the abdominal cavity, visceral fat is generally packed between the organs (e.g. stomach, liver, intestines, kidneys, etc.); around the heart; around the kidneys; and around the joints.

Alternatively, a brown adipose cells can be grown and differentiated in vivo in the subject. In this embodiment, one or more drug delivery systems containing an effective amount of one or more soluble ASC recruitment factors and brown adipogenic differentiation-inducing factors are administered to a subject in need of treatment, such as a subject at risk of developing diabetes, a diabetic patient, or an over-weight or obese patient.

Preferably the drug delivery system contains both the ASC recruitment factors and brown adipogenic differentiation-inducing factors and is administered in a single administration. In this embodiment, following administration to the desired site in the subject, the drug delivery system first releases the ASC recruitment factors, such as within 3 to 28 days, preferably 7 to 14 days following administration of the drug delivery system, and subsequently releases the brown adipogenic differentiation factors, such as after 3 to 28 days, preferably after 7 to 14 days following administration. Optionally two or more delivery systems are administered in two or more separate administrations, with the first drug delivery system containing the ASC recruitment factors. After a sufficient period of time, such as three to 28 days, preferably 7 to 28 days, more preferably 7 to 14 days, following the first delivery system administration, a second delivery system comprising the brown adipogenic differentiation factors is administered to the same site in the patient in an effective amount to induce differentiation of the ASCs into brown adipose cells.

### EXAMPLES

### Prophetic Example 1

### An Implantable, Nanoparticle-Polymeric Microfiber Mesh Trap for Recruiting and Containing Adipose Stem Cells

Adipose tissue-derived stem (ASC) cell isolation: ASCs will be enzymatically isolated from the subcutaneous abdominal fat or ZDF rats. Previous work has shown the multipotent capabilities of ASCs from this site (Guilak, F., et al. J Cell Physiol, 2006. 206(1):229-37; Fraser, J. K., et al. Trends Biotechnol, 2006. 24(4):150-4; Estes, B. T., et al. Nat Protoc, 2010. 5(7):1294-311). In each case, excised adipose tissue will be washed in sterile PBS and digested with collagenase type I (Worthington Biochemical, Lakewood, N.J.), and the released stromal cells isolated by density centrifugation. The cells will be expanded for three passages. In this manner, one is able to retrieve more than 400,000 ASCs per mL of original harvest tissue (human). For cellular expansion, ASCs will be washed twice with calcium and magnesium-free Dulbecco's Phosphate Buffered Saline (GibcoBRL, Gaithersburg, Md., USA) to remove media residue. Cells will be detached from the culture flask using trypsin-EDTA, then washed with DMEM/F12 and centrifuged at 500×g for 8 minutes. The cells will be re-suspended in DMEM/F-12, counted, and viability assessed using the trypan blue exclusion assay.

Identification of ASCs by FACS: Cells are prepared as a single cell at approximately 1×10⁷ cells/ml suspended in ice cold PBS with 10% FBS (Invitrogen, Carlsbad, Calif., USA) and 1% sodium azide (Sigma, St. Louis, Mo., USA) just prior to indirect immunofluorescence staining for surface markers, and are counted using a hemocytometer to determine total cell number. For each marker, 100 μl of cell suspension is added to a 1.5 ml centrifuge tube. 2 μg/ml of each primary antibody (e.g. ms IgG anti-CD34 and rb IgG anti-CD105, Abcam, Cambridge, Mass., USA) in 3% BSA/PBS is added to the suspension. The cells are incubated for 30 min at 4° C. in the dark. Cells are then washed thrice by centrifugation at 200 g for 5 min and resuspend again in ice-cold PBS. The fluorescently labeled secondary antibody is prepared in 3% BSA/PBS at the indicated concentration (e.g. 1 μg/ml of AlexaFluor 488-labeled donkey anti-mouse IgG and 2 μg/ml AlexaFluor 568-labeled donkey anti-rabbit IgG, Invitrogen) and incubate for 30 min at 4° C. The cells are washed three times in PBS by centrifugation at 200 g for 5 min and resuspended in ice cold 3% BSA/PBS with 1% sodium azide and stored in the dark for sorting.

Culture of ASCs in vitro: ASCs are cultured under aseptic, mammalian cell culture conditions in maintenance media (DMEM/F-12 (GibcoBRL), 10% FBS (Sigma), and 1× penicillin/streptomycin (GibcoBRL)) and, to confirm multipotency, clonally expanded and differentiated in each of chondrogenic induction media, osteogenic induction media, or adipogenic induction media. Maintenance media contains DMEM/F-12 (GibcoBRL), 10% FBS (Sigma), and 1× penicillin/streptomycin (GibcoBRL). Chondrogenic induction media contains DMEM-HG (GibcoBRL), 10% FBS, 1× penicillin/streptomycin, 1× ITS+ supplement (Collaborative Biomedical, Becton Dickinson, Bedford, Mass.), 110 mg/L sodium pyruvate (Sigma), 37.5 mg/mL ascorbate 2-phosphate (Sigma), 100 nM dexamethasone (Sigma), and 10 ng/mL TGF-β1 (R&D Systems, Minneapolis, Minn.). Osteogenic induction media contains DMEM-HG, 10% FBS, 1× penicillin/streptomycin, 10 mM β-glycerophosphate, 0.15 mM ascorbate-2-phosphate, 10 nM 1,25-(OH)₂ vitamin D₃, and 10 nM dexamethasone (Sigma). Adipogenic induction media contains DMEM/F-12, 3% FBS, 33 μm biotin, 17 μM pantothenate, 1 μM bovine insulin, 1 μM dexamethasone, 0.25 mM isobutylmethylxanthine (IBMX) (Sigma) (Guilak, F., et al. J Cell Physiol, 2006. 206(1):229-37).

Phenotype verification. Differentiated stem cell populations will be assayed using standard criteria as described (Guilak, F., et al. J Cell Physiol, 2006. 206(1):229-37; Elabd, C., et al. Biochem Biophys Res Commun, 2007. 361(2):342-8; Darling, E. M., et al. J Biomech, 2008. 41(17825308):454-464; Elabd, C., et al. Stem Cells, 2009. 27(11):2753-60; Estes, B. T., et al. Nat Protoc, 2010. 5(7):1294-311). Chondrogenesis will be evaluated by Toluidine Blue staining and immunohistology for identifying the presence of collagen II. Osteogenesis will be evaluated using alkaline phosphate activity and Alizarin Red staining. Adipocytic populations will be fixed with 10% formalin and then stained with Oil Red O (ORO, 0.5%) diluted 3:2 in isopropanol. Fraction of staining will be used to determine whether differentiation was successful. Adipogenesis will also be evaluated by leptin secretion, which will be quantified using a Human Leptin Quantikine ELISA kit (R&D Systems, Inc., Minneapolis, Minn.). Real-time PCR can also be used to further verify the upregulation of phenotype-specific genes for all conditions (chondrogenesis: collagen II, aggrecan; osteogenesis: osteopontin, osteocalcin; and adipogenesis: leptin, adiponectin).

Microarray analysis for establishing cell population multipotency: GEArrays from SuperArray Bioscience Corporation will be used to evaluate the presence and relative expression levels of select chondrocytic, osteoblastic, and adipocytic genes to verify isolated cell multipotency. In particular, ostepontin, osteocalcin, collagen II, aggrecan, leptin, and adiponectin expression will be examined in differentiating ASCs. 18S, GAPDH, and β-actin will be used as controls. Additional genes can be included as necessary. GEArrays function by binding DNA fragments to a nylon membrane matrix that has been modified with the genes of interest (Chan, B. P., et al. Biotechnol Bioeng, 2004. 88(6):750-8). Target labeling allows chemiluminescent imaging of the surface. Relative gene expression levels can be determined by normalizing to controls.

Fabrication of PDFG-BB, TGF-β, and SDF-1, and BSA (control) nanospheres: Nanospheres are fabricated with 50:50 poly (DL-lactide-co-glycolide, MW=12,000) (Boehringer Ingleheim Inc. Germany) using a novel phase inversion technique: phase inversion nanoencapsulation (PIN), developed in our laboratory. Briefly, a 50% solution of human recombinant PDFG-BB, TGF-β, SDF-1 or BSA (Chemicon) is combined with 10% bovine serum albumin and 10% Tween-20. This solution is added to a 0.001% polymer ethyl acetate solution and the two-phase system vortexed and immediately shell-frozen, cooled in liquid N₂ followed by lyophilization for 48 hours. The dried polymer product is re-suspended in ethyl acetate (4% (w/v)) and the solution rapidly poured into petroleum ether (Fisher) for formation of nanospheres that are filtered and lyophilized for 48 hours for final solvent removal.

Unencapsulated growth factor (PDFG-BB, TGF-β, and SDF-1) controls: Unencapsulated growth factors are included as controls. The total dose of each growth factor delivered over 21 days will be calculated from release profile data. The total calculated dose is injected into the sterilized blank nylon mesh pouch immediately following implantation.

Nanosphere-mesh Implant fabrication: 0.8 cm×0.8 cm squares of nylon mesh; Spectrum Labs, Irving, Tex., USA) with a pore size of 20 microns are heat sealed on three sides and sterilized (Amsco Gravity 2051 autoclave). Appropriate nanospheres are added to each “bag” and the fourth side heat-sealed prior to surgery.

TABLE 1
List of groups for surgical implantation and harvesting (n = 6).

| Control Groups | Experimental Groups |
|---|---|
| Blank Nylon Mesh | Mesh with PDGF-BB nanospheres |
| Mesh with BSA nanospheres | Mesh with SDF-1 nanospheres |
| Mesh with lyophilized | Mesh with TGF-β nanospheres |
| PDGF-BB | |
| Mesh with lyophilized SDF-1 | Mesh with PDGF-BB & SDF-1 |
| | nanospheres |
| Mesh with TGF-β | Mesh with PDGF-BB & TGF-β |
| | nanospheres |
| | Mesh with SDF-1 & TGF-β nanospheres |
| | Mesh with PDGF-BB, SDF-1, & TGF-β |
| | nanospheres |

Surgical introduction of mesh implants and controls, in vivo: Nylon pouches containing nanospheres will be implanted subcutaneously into the subcutaneous abdominal fat of 9 week old male Zucker Diabetic Fatty (fa/+, lean) rats. The mesh pore size ranges between 15 and 20 microns in diameter and the total implant comprises two 0.8 by 0.8 cm pieces of porous nylon heat-sealed at the margins. These pouches will be filled with either the appropriate number of nanospheres or the appropriate amount of lyophilized control protein. Groups included: implant only, implant containing lyophilized protein, implant with plain PLA and PLGA nanospheres, and implants loaded with nanospheres containing either one or a combination of PDGF-BB, SDF-1 or TGF-13 (Table 1). The rat is anesthetized in an asphyxiation chamber with administration of inhalational isofluorane®. Anesthesia will be maintained throughout the procedure by the administration of inhalational isofluorane® via a nose cone. A 1 cm incision will be made into the abdominal skin using a scalpel equipped with a number 11 blade. The incised skin will be separated from the underlying adipose and facial tissue by scissor spreading. The recruitment factor-eluting nanosphere-nylon mesh stem cell trap will be placed subcutaneously and tacked in place with one interrupted subcutaneous 4-0 nylon suture towards the periphery of the implant. After implant placement, the wound is closed using running resorbable sutures (Vicryl 6-0). After 7 or 21 days, animals will be sacrificed using an overdose of metofane. Implants and adjacent tissue will be immediately removed, placed in OCT embedding medium (Sakura Finetek Inc. Torrance, Calif., USA) and quick-frozen on dry ice for storage at ⁻80° C. until further analysis.

Terminal harvest of implants and verification of ASC recruitment by in situ Immunofluorescence staining: Rats will be sacrificed at days 3, 7 and 14 (n=2 per group), and the tissue quickly frozen in OCT embedding media (Sakura Finetek Inc) and stored at ⁻80° C. until immunohistochemical analysis. The ability of implants to recruit progenitor cells over time is assessed via immunostaining for UCP1, CD34, and CD105. Briefly, frozen sections are brought to room temperature, OCT embedding medium dissolved in PBS (Sigma) and the tissue fixed in either 2% paraformaldehyde (Electron Microscopy Sciences, Warrington, Pa., USA) for 10 minutes or acetone at −20° C. for 2 minutes. Sections are blocked with 4% bovine serum albumin (Sigma Chemical) and 10% goat serum (Jackson ImmunoResearch Laboratories, Inc., West Grove, Pa., USA) for 1 hour. The primary antibodies diluted appropriately in blocking solution are applied for 1 hour at room temperature in a humidified chamber. The sections are then rinsed and blocked with 4% BSA/10% goat serum for 1 hour. Corresponding secondary antibodies are applied for 45 minutes at room temperature (e.g. Alexa 647 nm, Alexa 488 nm, Alexa 568-conjugated all from Molecular Probes, Oregon). All sections are either mounted in PBS or counterstained using DAPI to visualize nuclei (Slow Fade mounting media, Invitrogen). Stained sections are analyzed with a confocal laser scanning microscope (Zeiss 410, Thornwood, N.Y., USA) or a fluorescence/light microscope (Zeiss Axiovert 200M Light Microscope). At least 4 areas on stained slides stained are captured for image analysis at 25×. Analysis will be conducted at a distance of up to 400 μm from the implant perimeter. Scion Image analysis Beta 4.0.2 (NIH software) is used to assess captured images.

### Prophetic Example 2

### In Vitro Culture Methodology for Efficiently Inducing Brown Adipogenic Differentiation of ASCs

A broad array of factors will be screened for their capacity to induce brown adipogenic differentiation of adult human and rat ASCs in a well-plate format. Cell differentiation/phenotype will be characterized first by immunofluorescence staining for the brown adipocyte marker UCP1, then verifying phenotype of cells from positively screened conditions by RT-PCR and Oil Red-O Staining for multilocular fat globes characteristic of brown adipocytes, but not their white counterparts.

ASCs from adult human lipoaspirate and from subcutaneous abdominal fat of lean (fa/+) male Zucker Diabetic Fatty rats (Charles River Labs, Wilmington, Mass., USA) will be exposed to combinations of the brown adipogenic differentiation-inducing factors, in particular Bone morphogenetic protein 7 (BMP7) (Tseng, Y. H., et al. Nature, 2008. 454(7207):1000-4; Guo, X. and K. Liao. Gene, 2000. 251(10863095):45-53), cyclic AMP (CAMP) (Klaus, S. Bioessays, 1997. 19(3):215-23), retinoic acid (RA; low concentrations) (Alvarez, R., et al. J Biol Chem, 1995. 270(10): p. 5666-73), triiodothyronine (T3) (Darimont, C., et al. Mol Cell Endocrinol, 1993. 98(1):67-73; Obregon, M. J. Thyroid, 2008. 18(2):185-95), dexamethasone (Dex) (Zilberfarb, V., et al. Diabetologia, 2001. 44(3):377-86; Klaus, S. Bioessays, 1997. 19(3):215-23; Freake, H. C. and Y. K. Moon. J Nutr Sci Vitaminol (Tokyo), 2003. 49(1):40-6), growth hormone (GH) (Guo, X. and K. Liao. Gene, 2000. 251(10863095):45-53; Shang, C. A., et al. Cell Endocrinol, 2002. 189(1-2):213-9), insulin (Klaus, S. Bioessays, 1997. 19(3):215-23; Fasshauer, M., et al. Mol Cell Biol, 2001. 21(1):319-29), and insulin-like growth factor 1 (IGF-1) (Benito, M., et al. Int J Biochem Cell Biol, 1996. 28(5):499-510), in ASC maintenance and adipogenic differentiation media, as described above.

Characterization of cellular differentiation will be conducted via three approaches: preliminarily, during the high-throughput screen, by indirect immunofluorescence staining of fixed cells in culture for UCP1 and PRDM16 expression, 2) then candidates by Q-RT-PCR analysis for brown fat specific markers (PRDM 16, PGC-1α, and PGC-1β), as well as 3) oil red O staining for multilocular fat in cells with dye extraction to quantify lipid content per sample (Guilak, F., et al. J Cell Physiol, 2006. 206(1):229-37; Wickham, M. Q., et al. Clin Orthop Relat Res, 2003(412):196-212).

mRNA quantitation by RT-PCR: Murine mRNA levels for the genes of interest (UPC1,) will be determined by RT-PCR with a real-time PCR machine from Roche (LightCycler™). If necessary, additional genes can be investigated to track differentiation towards the different cell lineages. Total RNA will be isolated with the Qiagen “RNeasy” kit, a procedure that includes DNAse treatment. For each sample, commercially-available primers will be used for PCR amplification and detection. 18S primers and probes will be added to each sample to provide an internal control for the RNA isolation/DNase, RT, and PCR steps. HPLC-purified primers (GibcoBRL) will be used for PCR. A standard curve for the genes of interest will be created by serial dilution of a known quantity of each PCR product. The standard curve and the amount of each cDNA will be calculated based on the cycle number at which the second derivative maximum of fluorescence intensity occurs, detected by SYBR green. Results will be expressed as a ratio of the mRNA of gene of interest (e.g., collagen) to the mRNA of 18S. The specificity of PCR reactions will be monitored by the melting curve analysis and by gel electrophoresis of selected samples (Erickson, G. R., et al. Biochemical & Biophysical Research Communications, 2002. 290(2):763-9; Wickham, M. Q., et al. Clin Orthop Relat Res, 2003(412):196-212).

### Prophetic Example 3

### In Vivo Transplantation of ASC-Derived Brown Adipocytes

To date, no procedure exists that enables a physician to increase autologous brown adipose tissue mass. The efficacy of an ASC-derived brown adipose cell replacement therapy will therefore be pre-clinically evaluated in an animal model of obese diabetics. In ZDF rats, a mutation in the leptin receptor, OB-R, is associated with leptin resistance, obesity, and increased fat content of islets. The leptin receptor mutation in Zucker Diabetic Fatty (ZDF) rats consists of a G1u269 to Pro in the extracellular domain. This alters post-receptor signal transduction so that leptin resistance and obesity develop. Increased nitric oxide (NO) generation, due to high intracellular levels of long-chain fatty acids, impairs β-cell function and prevents their compensation for adipogenic diabetes (Unger, R. H. Trends Endocrinol Metab, 1997. 8(7):276-82), providing a model for investigating the therapeutic potential of BAT for treating obesity and obesity-influenced diabetes. The outcomes of such a novel, brown fat transplantation study could open up new avenues in the fields of obesity and diabetes research, as a cell-implantation based approach to metabolic enhancement has yet to be demonstrated in the literature.

Proof of concept for an autologous ASC-derived brown adipose cell replacement therapy will be provided utilizing: PAZ6 brown adipocytes for proof of principle; and ASCs harvested from the subcutaneous abdominal fat of lean (fa/+) male (10 weeks) ZDF rats from Example 1. With the human brown adipocyte cell line PAZ6, transplantation will be attempted in immune-competent animals; however, if graft rejection is apparent, drug-induced immunosuppression (e.g. Tacrolimus (FK 506) (Tanaka, M., et al. Transplant Proc, 1996. 28(2):679-80)) will be incorporated into the protocols. For cells from AIM 1, given ZDF rats are an inbred strain, syngeneic transplantation of these ASC-derived brown adipose cells will be approached. In each case, studies will be conducted in obese (fa/fa) male age-matched (10 weeks) ZDF rats, maintained in metabolic cages, monitored for weight loss and markers of diabetes over 2 months (untreated rats reliably develop diabetes by week 12 on a controlled diet of Purina #5008). Sham injection (n=6) using PBS without cells into lean (fa/+) age-matched male ZDF rats will be made for comparison.

Maintenance of Zucker Diabetic Fatty (ZDF) rat model: On non-experimental days, rats are housed in individual metabolic cages and allowed access to rat chow and water. Keto-diastix test strips (Baxter) are used for the detection of glycosuria and ketonuria. A diagnosis of diabetes is made when glucose is detected in the urine (glycosuria) and when a blood glucose concentration exceeding 250 mg/dL is observed. Rats will be subcutaneously injected with protamine zinc insulin (PZI) U-40, a combination of beef/pork insulin, obtained from Blue Ridge Pharmaceuticals, Inc. at approximately noon every day. Because PZI has a 12-24 hour duration of action, the injections will be made to coincide with the rats' feeding time. This ensures that blood glucose levels will not decrease to hypoglycemic levels prior to the rats consuming enough food to balance the insulin injection. Additionally, rats will be weighed daily.

ASC-derived brown adipose cell injection protocol: For all diabetic rat experiments, rats will be first anesthetized in a 4% isoflurane gas chamber. Rats are then placed on nosecones and maintained on 1-2% isoflurane for the initial blood sample which was taken via tail bleed. Brown adipose cells are prepared as single cell suspensions in sterile PBS at approximately 5×10⁶ cells/ml and injected into the abdominal fat in five different locations, with volumes of 200 μl per injection through a 21 gauge beveled syringe needle. Animals are then maintained in metabolic cages for the remainder of the experiment with weight, blood and urine glucose, and blood plasma insulin quantified at intervals as described above. Blood Analysis: Blood samples are taken from tail bleeds at serial points postoperatively using rat restraint tubes while the rats were conscious. Blood will be collected in heparinized tubes, spun down and the plasma recovered for glucose and insulin analysis. A glucose trinder assay (Diagnostic Chemicals Limited, Oxford, Conn.) will be used to determine plasma glucose levels for the rat experiments. For the detection of endogenous insulin in the plasma of experimental rats, an insulin ELISA will be used (Diagnostic Systems Laboratories, Webster, Tex., USA).

Detection of glucose and insulin in vivo: The Glucose Trinder assay from Diagnostic Chemicals Limited (Oxford, Conn.) will be used to determine plasma glucose levels (PGL), and Keto-diastix test strips (Baxter) will be used for the detection of glucose in urine in experimental animals, as above. In addition, an ELISA (an enzymatically amplified ‘one-step’ sandwich-type immunoassay) kit from Diagnostic Systems Laboratories (Webster, Tex., USA) will be used to detect insulin in blood plasma collected as described above.

1. A drug delivery system for recruiting adipose stem cells (ASCs) to a site in the body of a subject, wherein the system comprises a plurality of particles, fibers, or films comprising one or more soluble ASC recruitment factors releasably incorporated therein, wherein the one or more ASC recruitment factors are released from the drug delivery system when implanted in a subject in an effective amount to recruit ASCs.

2. The drug delivery system of claim 1, wherein the system comprises a plurality of fibers or films.

3. The drug delivery system of claim 1, wherein the one or more soluble ASC recruitment factors are selected from the group consisting of SDF-1, PDGF-BB, and TGFβ.

4. The drug delivery system of claim 1, wherein an effective amount of ASC recruitment factors is released from the drug delivery system for at least 14 days following implantation in a subject.

5. The drug delivery system of claim 1, further comprising an external porous housing having pores of a size sufficient to allow migration of ASCs into the system.

6. The drug delivery system of claim 5, wherein the external porous housing is a polymeric mesh.

7. The drug delivery system of claim 6, wherein the polymeric mesh comprises one or more non-erodable polymers.

8. The drug delivery system of claim 6, wherein the polymeric mesh comprises one or more polymers selected from the group consisting of polyamides, polyethylene, polypropylene, polystyrene, polyvinyl chloride, polycarbonates, poly(amino acids), polyesteramides, poly(dioxanones), poly(alkylene alkylates), polyethers, polyurethanes, polyetheresters, polyacetals, polycyanoacrylates, polysiloxanes, poly(phosphazenes), polyphosphates, polyalkylene oxalates, polyacrylonitriles, polyalkylene succinates, poly(maleic acids), polysaccharides, poly(acrylic acids), poly(methacrylic acids), and derivatives, copolymers, and blends thereof.

9. The drug delivery system of claim 8, wherein the polymeric mesh comprises one or more polyamides.

10. The drug delivery system of claim 1, wherein the particles, fibers or films comprise one or more biodegradable polymers.

11. The drug delivery system of claim 10, wherein the biodegradable polymers are selected from the group consisting of polyhydroxyacids, polyhydroxyalkanoates, poly(caprolactones), poly(orthoesters), poly(phosphazenes), polyesteramides, polyanhydrides, poly(dioxanones), poly(alkylene alkylates), poly(hydroxyacid)/poly(alkylene oxide) copolymers, poly(caprolactone)/poly(alkylene oxide) copolymers, biodegradable polyurethanes, poly(amino acids), polyetheresters, polyacetals, polycyanoacrylates, poly(oxyethylene)/poly(oxypropylene) copolymers, and derivatives, copolymers, and blends thereof.

12. The drug delivery system of claim 11, wherein the polyhydroxyacid is selected from the group consisting of poly(lactic acid), poly(glycolic acid), and poly(lactic acid-co-glycolic acid).

13. The drug delivery system of claim 1, wherein the particles, fibers or films are electrostatic.

14. The drug delivery system of claim 1, wherein the particles have a mean diameter of from 10 nm to 10 μm.

15. The drug delivery system of claim 1, further comprising a second plurality of particles, fibers or films comprising one or more brown adipogenic differentiation-inducing factors releasably incorporated therein, wherein the one or more brown adipogenic differentiation-inducing factors are released from the drug delivery system when implanted in a subject in an effective amount to induce differentiation of ASC's into brown adipose cells.

16. The drug delivery system of claim 15, wherein the one or more brown adipogenic differentiation-inducing factors are selected from the group consisting of bone morphogenetic protein 7 (BMP7), cyclic AMP (cAMP), retinoic acid (RA), triiodothyronine (T3), dexamethasone (Dex), growth hormone (GH), insulin, and insulin-like growth factor 1 (IGF-I).

17. A method for isolating autologous adipose stem cells (ASCs) from a subject comprising:
(a) introducing into the subject a drug delivery system,
wherein the drug delivery system comprises a plurality of particles, fibers, or films comprising one or more soluble ASC recruitment factors releasably incorporated therein, wherein the one or more ASC recruitment factors are released from the drug delivery system when implanted in a subject in an effective amount to recruit ASCs,
(b) removing the drug delivery system from the subject after a sufficient time period for ASCs to migrate into the drug delivery system, and
(c) isolating the ASCs.

18. The method of claim 17, further comprising culturing the ASCs in the presence of an effective amount of one or more brown adipogenic differentiation-inducing factors to induce differentiation of the ASCs into brown adipocytes.

19. The method of claim 18, wherein the one or more brown adipogenic differentiation-inducing factors are selected from the group consisting of bone morphogenetic protein 7 (BMP7), cyclic AMP (cAMP), retinoic acid (RA), triiodothyronine (T3), dexamethasone (Dex), growth hormone (GH), insulin, and insulin-like growth factor 1 (IGF-I).

20. The method of claim 17, wherein the ASCs are CD45⁻/CD31⁻/CD34⁺/CD105⁺ cells.

21. The method of claim 18, further comprising administering to the subject an effective amount of the brown adipocytes for the treatment of obesity or diabetes.

22. (canceled)

23. A method for treating obesity or diabetes in a subject comprising introducing into the subject the a drug delivery system,
wherein the system comprises
(a) a first plurality of particles, fibers, or films comprising one or more soluble ASC recruitment factors releasably incorporated therein,
wherein the one or more ASC recruitment factors are released from the drug delivery system when implanted in a subject in an effective amount to recruit ASCs, and
(b) a second plurality of particles, fibers or films comprising one or more brown adipogenic differentiation-inducing factors releasably incorporated therein,
wherein the one or more brown adipogenic differentiation-inducing factors are released from the drug delivery system when implanted in a subject in an effective amount to induce differentiation of ASC's into brown adipose cells.

24. A kit comprising the drug delivery system of claim 1 and one or more brown adipogenic differentiation-inducing factors.

25. The kit of claim 24, wherein the one or more brown adipogenic differentiation-inducing factors are selected from the group consisting of bone morphogenetic protein 7 (BMP7), cyclic AMP (cAMP), retinoic acid (RA), triiodothyronine (T3), dexamethasone (Dex), growth hormone (GH), insulin, and insulin-like growth factor 1 (IGF-I).
